(19) United States
(12) Patent Application Publication
(10) Pub. No.: US 2026/0102099 A1
(43) Pub. Date: Apr. 16, 2026

(54) TUNABLE CATHETER

(71) Applicant: COREMAP, INC., Burlington, MA (US)

(72) Inventor: Peter S. Spector, Burlington, MA (US)

(21) Appl. No.: 19/357,768

(22) Filed: Oct. 14, 2025

Related U.S. Application Data
(60) Provisional application No. 63/707,396, filed on Oct. 15, 2024.

(51) Int. Cl. A61B 5/287; A61B 5/00; A61B 5/361

(52) U.S. Cl. CPC A61B 5/287; A61B 5/361; A61B 5/6852; A61B 2560/0468; A61B 2562/046

(57) ABSTRACT

The present disclosure applies to methods of cardiac ablation that use catheters able to conform and adapt to the heart of a patient in the middle of surgery. The catheters comprise paddles at the distal ends that have an array of electrodes on at least one side. The durometer of the paddles can be changed through physical manipulation of wires attached to the paddles or through other means such as the material the paddles are made of. Electrodes on the paddle record cardiac signals and a computer analyzes these signals to determine where in the heart fibrillation is originating. The computer then outputs a site for ablation.

### FIELD OF INVENTION

The present disclosure applies to systems and methods of cardiac ablation that use catheters able to conform and adapt to the heart of a patient in the middle of surgery.

### BACKGROUND

Cardiac fibrillation is an uncontrolled twitching of muscle fibers (fibrils) resulting in an irregular and often rapid heart arrhythmia associated with increased mortality and risk of stroke and heart failure. See, e.g., Calkins et al., Treatment of Atrial Fibrillation with Anti-arrhythmic Drugs or Radio Frequency Ablation: Two Systematic Literature Reviews and Meta-analyses, 204) Circ. Arrhythmia Electrophysiol. 349-61 (2009). Cardiac fibrillation manifests as a loss of normal electrical coordination in the heart that can result in sudden cardiac death. Existing treatments for cardiac fibrillation include medications and other interventions to try to restore and maintain normal, organized electrical activity. When medications, which are effective only in a certain percentage of patients, fail to maintain normal electrical activity, clinicians may resort to incisions made during open heart surgery or minimally invasive ablation procedures, whereby lines of non-conducting tissue are created across the cardiac tissue in an attempt to interrupt the circuits of electrical excitation to include only organized activity and not fibrillation. Id. at 355. If sufficient and well-placed, the non- conducting tissue (e.g., scar tissue) will interfere with and normalize the erratic electrical activity.

However, part of the success of current ablation techniques depends on the catheter. Existing catheters have several shortcomings, including that the electrodes are too large, the inter-electrode spacing is too great or having to move the catheter to different locations in the heart – a technically challenging process. There remains a need for adaptable catheters to achieve the desired clinical benefit offered by ablation treatments.

### SUMMARY OF THE INVENTION

The present invention provides methods and systems for catheter ablation using a unique catheter with a distal sensor able to conform to cardiac tissue by being manipulated while it is in the heart. The catheter is comprised of electrodes on a paddle with tunable conformance that records signals from the cardiac tissue surface. The signals are then entered into a computer that has a program capable of determining a locus important to the propagation of cardiac fibrillation in the cardiac tissue. The program then outputs a site for ablation to attempt to interrupt the propagation.

In a preferred embodiment, a catheter of the invention comprises an electrode array that conforms to the cardiac tissue, allowing the physician to construct a high-resolution map of the cardiac tissue. The contact of the electrode arrays against tissue allows for a consistent, stable signal that reduces noise and allows for more accurate mapping of bioelectrical activity. More accurate mapping provides the ability to characterize electrophysiological properties of the tissue in order to determine a precise location for ablation to treat cardiac fibrillation.

Catheters of the present invention can be tuned in various ways while in use. In a preferred embodiment, the paddle of the catheter is connected to one or more wires. The wires can be connected on the outside of the paddle or can be inserted into the paddle so that they are seated between the two faces of the paddle. In that embodiment, the wires are physically controlled by a physician. In certain embodiments, the paddle contains a single wire and in other embodiments there are a plurality of wires. If there is more than one wire present, the durometer of the catheter may be manipulated by moving the wires relative to each other (e.g., moving the wires closer together make the catheter more flexible, whereas moving them further apart makes the catheter stiffer). Different wires of varying durometer can also be used to change the stiffness of the catheter. In the simplest example, a single wire of the desired durometer can be inserted into the paddle, nested between both faces of the paddle, in order to adjust the durometer of the catheter.

A catheter of the invention is tuned to a desired durometer via the use of specific materials. For example, the catheter can be composed of an electroactive polymer the durometer of which changes based on an electric field, electric current, voltage, or electrical waveform thereof, or a local temperature change based on an applied electricity. Suitable materials include specialty materials such as various dielectric elastomers, conducting polymers such as polyaniline, polypyrrole, or polythiophene, composite materials made from metals and polymers, piezoactive polymers such as polyvinylidene fluoride, or formulations of conventional materials such as polyurethane, Teflon, or polyamide. Based on the achievable durometers of a material, the catheter may be made with multiple layers that can include just one material or a combination to achieve the desired range of conformance for the catheter.

Tuning the catheter by changing the durometer has various other effects on the catheter. For example, changing the durometer can cause the catheter to lie flat, allowing it to conform to the shape of the cardiac tissue. Changing the durometer can also change the density of the electrodes on the paddle (e.g., if the catheter has a lower durometer i.e., is less stiff, then the electrodes might be denser in one area compared to another. In contrast, if the durometer is increased i.e., the catheter is stiffer, then the electrodes will be more spaced out).

Once the desired durometer has been reached and the paddle of the catheter is against the tissue surface, the electrodes can record cardiac signals from the surface of the tissue. The electrodes on the paddle may be in an orthogonal, close, and unipolar (OCU) arrangement relative to the tissue surface. The cardiac signals detected by the electrodes are then entered into a computer that has a program capable of determining “driver sites”: loci important to the propagation of fibrillation in the cardiac tissue. The computer then outputs a site within the cardiac tissue for ablation.

### BRIEF DESCRIPTION OF THE DRAWINGS

FIG. 1 shows a catheter with a paddle and an array of electrodes.

### DETAILED DESCRIPTION

The present invention provides methods and systems for using a tunable catheter that can conform to the shape of cardiac tissue and that can be tuned while in use. Preferred catheters comprise a paddle with an array of electrodes to detect and record cardiac signals. These cardiac signals are then input into a computer comprising software and associated hardware to analyze the signals and determine a locus of atrial fibrillation, enabling focused ablation.

Devices and methods taught herein accurately monitor bioelectrical signals in cells, tissues, and/or organs for the purposes of tissue property mapping. Devices of the present invention achieve high temporal and spatial resolution (including improved signal-to-noise ratio) which improves the understanding of the dynamic behavior of the electrophysiological properties of the tissue. The result is accurate mapping of tissue properties, including, but not limited to, voltage, activation time, cycle length, conduction velocity and refractory period. In the case of atrial fibrillation, methods and devices of the invention enable a more targeted, less extensive ablation strategy as compared to conventional ablation strategies.

Persistent atrial fibrillation is known to be correlated with shortened cycle length in the atria. By accurately mapping cycle length and conduction velocity and characterizing regional gradients in the atria, optimal target sites for ablation are identified to terminate persistent atrial fibrillation, while sparing tissue from excessive ablation. Treatment of cardiac arrhythmias by catheter ablation includes identifying an area of cardiac tissue having aberrant electrically conductive pathways and then using applied energy to create lesions or scars to isolate or modify the tissue believed to be the source of the arrhythmia. This procedure blocks the abnormal electrical signals. For catheter ablation to be successful, the tissue where the faulty electrical activity occurs must be accurately identified.

Physicians commonly perform cardiac mapping with catheters that are introduced percutaneously in the heart chambers and which sequentially record the endocardial electrograms. Catheters are small flexible tubes capable of insertion into a blood vessel, for example in the groin, arm or neck, and then threaded into the heart. Cardiac mapping is the process of identifying the temporal and spatial distributions of myocardial electrical potentials during a particular heart rhythm in order to determine the cause of that rhythm. Substrate abnormalities that cause the arrhythmia may manifest as variations in conduction velocity, minimum cycle length, and/or other measurable properties derived from intracardiac signals. Therefore, analysis of signals may be applied in order to deduce tissue property characteristics and arrhythmia driver sites.

Mapping is performed using electrophysiological signals originating from muscular, cardiac or neurological activity. These signals that can be measured through changes in electrical potential across a cell, tissue, or organ.

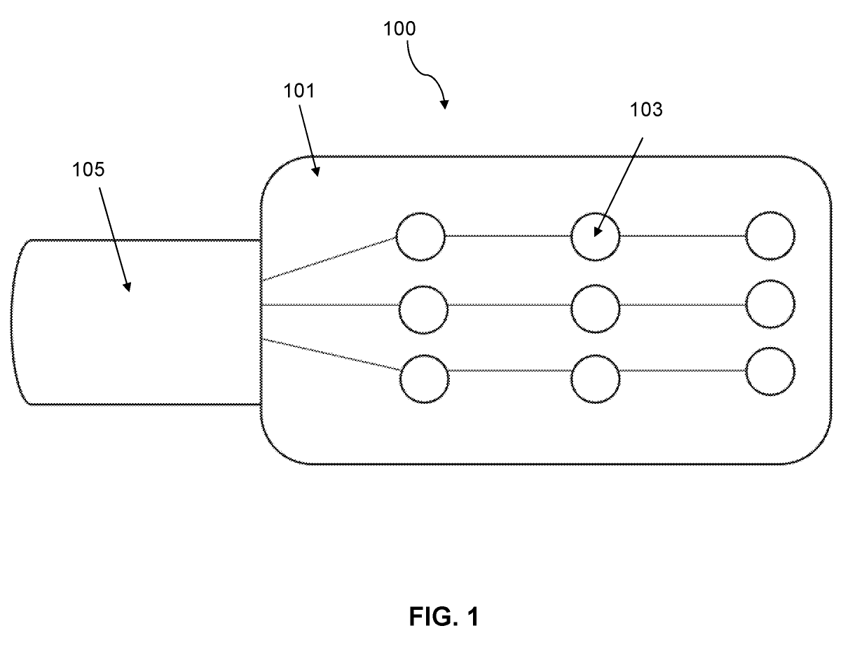

FIG. 1 shows an embodiment of the present invention. The catheter 100 is comprised of a paddle 101 located on the distal end of the catheter 100 with an array of electrodes 103 on the paddle. Electrodes 103 may be on one side or on both sides of the paddle 101. Connected to the paddle 101 is a shaft 105 that allows a physician to guide the catheter 100 into the heart and inside the shaft (or otherwise incorporated into the shaft) is a wire to tune the conformance of the paddle 101 to the cardiac tissue.

### Tunability

The paddle on the catheter can be manipulated in a number of ways that will be detailed below. The benefit of a catheter with tunable conformance is that it allows the electrodes on the catheter to lie flat against the surface of the cardiac issue giving more consistent and clear recordings of cardiac activity. Additionally, the catheter can be manipulated while inside of the patient's heart.

### Wires

The shape of the paddle can be manipulated physically using wires. The wires can be any medical-grade wires known in the art. The wires can be connected to the paddle on the outside or, preferably, the wires can be nested inside of the paddle. A physician can manipulate the wires in a number of ways which will affect the conformance of the paddle. For example, if there are more than one wire inside of the paddle, then a physician can pull the wires from the proximal end of the catheters, which are arranged to move further apart from each other in the paddle, leading to decrease in the conformance, (i.e., stiffness) making the paddle more taut. If a physician pulls on the wires in a manner that moves the wires closer together in the paddle, then the ability to conform increases, and the catheter becomes more flexible or less taut. In this preferred embodiment, the physician can tune the conformance of the catheter throughout the procedure without ever having to take the catheter out of the patient's heart.

The paddle on the catheter can also be manipulated by using wires of various stiffnesses. For example, a single wire that has a width similar to the paddle can be inserted into the paddle, nested between the two faces of it. Thus, the paddle's ability to conform will be directly dependent on the stiffness of the wire inserted (e.g., a wire that is of medium rigidity leads to a catheter with a paddle that is also of medium rigidity).

### Material

Another embodiment of the invention includes changing the material the paddle is made of. A non-exhaustive list of material that a paddle can be made of includes polyurethane, Teflon, polyamide, or an electroactive polymer. The electroactive polymer can change properties, such as durometer, based on an electric current that is run through it. In application, a physician can control the electric field, electric current, voltage, electrical waveform thereof, or a local temperature change based on a electricity applied to the paddle from a computer or system that is connected to the catheter while the catheter is inside the patient's heart.

Durometer can also be controlled by layering materials. A non-exhaustive list of materials that can be used for the paddle include silicone, polyimide, PEEK 450G, nylon, Pebax or Pellethane. Layers can be the same material throughout or can differ at each layer. For example, the paddle may have a first layer with PEEK or a thermoplastic polyurethane having a durometer number of 87D, and a second layer of Pebax having a durometer number of 55D.

The layers may also cover the entirety of the paddle or only some of it. For example, the second layer material may then form a web around the first material. In some embodiments, the first layer may cover one entire face of the paddle, while the second layer may form a web-like structure on top of the first layer, or vice-versa. The web structure may appear as open interstices or may resemble a web, netlike, or netted pattern.

The durometer of the paddle may also be changed by attaching to one side of the paddle (a side without electrodes) a piece of one of the above materials that is the same shape and size of the paddle. This piece of material can be attached with an adhesive and changed depending on the durometer needed for the procedure.

### Temperature and Ultraviolet Light

The durometer of the paddle can also be changed by controlling the temperature of it. For example, the paddle can be made with or attached to a material whose durometer is temperature dependent where warming the material can cause it to be more flexible or cooling it can make it stiffer. The material can also be a medical-grade resin that gradually stiffens as it is cured under ultraviolet light.

### Electrodes

The present disclosure provides systems and methods using electrode arrays on a paddle that can be positioned within a patient's heart. An electrode is an electrical conductor. The electrodes may comprise two or more materials. For example, the electrodes may be constructed from conductive layers such as copper and gold, iridium oxide coatings to achieve certain electrode properties and one or more insulating layers over the portions of the conductors not meant to contact tissue. In some embodiments, individual sensing electrodes include a copper trace and a copper pad that may are plated with gold. The gold thus covers the copper components of the electrode to prevent the copper from leaching into solution. The electrode may then be further coated with iridium oxide to reduce the impedance and to improve signal-to-noise ratio. The gold plating acts as a corrosion inhibitor for the copper trace. In some embodiments, the electrode comprises a slot below the insulating layer into which the copper trace is embedded such that the gold plating is on top of the copper electrode.

The electrodes may be manufactured using methods known in the art such as by using printed circuit techniques followed by coating with iridium oxide using photoresist techniques.

In some embodiments, the electrodes of the invention are microelectrodes shaped substantially as thin discs. The electrodes, as thin discs, may be positioned as a contiguous array on the surface of the paddle. The electrodes may be positioned, as shown in, for example, FIG. 1. Notably, the height, or alternatively the thickness, of the electrode may be less than about 150µm. In some embodiments, the height of the electrode is less than about 25µm. Height, as used herein, means the height of the electrode as measured from the bottom of the electrode relative to its orthogonal position on the tissue to the top of the electrode. Alternatively, the height of the electrode may also refer to the thickness of the electrode as a thin disc. In non-limiting embodiments, the diameter of the electrodes may be 150µm such that the electrode surface area is less than or equal to 0.01767 mm2.

### Unipolar, Bipolar, and OCU Arrangements

There are generally two electrode types: unipolar and bipolar electrodes. Unipolar electrodes are the simplest arrangement, with one recording electrode within the heart and another at a relatively long distance away. Unipolar electrodes are adequate but have a tendency to include far field electrical activity in the recorded signal which can result in a fractionated electrogram. This is a particularly relevant when trying to map complex arrhythmias (e.g., cardiac fibrillation) and accurately identifying local activation time. Bipolar electrode arrangements ameliorate this problem by placing both electrodes within the heart at a relatively narrow distance apart. Since both electrodes "see" approximately the same far field electrical activity and the recorded potential is the difference of the two, the resultant electrogram includes little far field signal. There are, however, at least two limitations of bipolar electrodes. First, the recorded electrical potential of bipolar electrodes vary with their orientation relative to the direction of a passing wavefront. Second, because bipolar electrodes usually have both electrodes on the heart surface, the primary electrical activity detected by each electrode is potentially arising from distinctly different areas of the heart, and would not be appropriate to subtract. In view of these limitations, it was hypothesized that bipolar electrodes oriented perpendicular to the tissue plane (orthogonal close unipolar (OCU)) retain the superior near/far-field discrimination of common bipolar electrode recordings with the directional independence and smaller footprint of unipolar recordings.

Electrodes of the present disclosure may be unipolar electrodes. In a pair of unipolar electrodes, a first electrode, the "index electrode", is proximal to tissue such that it records a signal. A second electrode, the "indifferent electrode" is positioned away from tissue such that it does not record a signal.

Electrodes of the present disclosure may be contact bipolar electrodes. In a pair of contact bipolar electrodes, both electrodes are proximal to tissue such that each records a signal.

Electrodes of the present disclosure may be arranged as orthogonal close unipolar (OCU) electrodes. In a pair of OCU electrodes, the index electrode and indifferent electrodes are in a stacked arrangement and positioned orthogonal to a tissue. The index electrode and indifferent electrode are separated by an inter-electrode space that is likewise orthogonal to the tissue. The inter-electrode space is preferably between approximately 0.01 mm and 1 mm. In one embodiment, the index electrode can be on one flat face of the paddle while the indifferent electrode is directly opposite the index electrode on the other flat face of the paddle.

OCU electrodes have been shown to be particularly effective in the context of mapping fibrillation. OCU electrodes retain the ability of contact bipolar electrodes to exclude far field electrical activity endemic to unipolar arrangements. Further, OCU electrodes retain the directional independence and small footprint of unipolar electrodes, which are lacking in contact bipolar electrodes.

### Electrode Density

By using an electrode array on a paddle of variable conformance, a variable recording site density can be achieved. Here, electrodes on the paddle are not evenly distributed but rather are concentrated with high density in one part of the catheter paddle and lower density at other locations on the paddle. This can be achieved by varying the conformance of the paddle in ways mentioned above. In a preferred embodiment, wires are placed in the paddle and extend out such that a physician can physically manipulate them. In doing so, the conformance can be changed based on the placement of the wires relative to each other (e.g., if the wires are closer together, then the paddle will have a greater ability to conform and be less taut, causing the electrodes to be less spread out and more focused i.e., dense in one area. The reverse it true if the wires are further apart, leading to reduced ability to conform and electrodes that are more spaced out. By having a paddle with tunable conformance, there is no need to change the catheter size or shape and one can focus density in one region while simultaneously having a broad view of other areas.

### Cardiac Signal Detection

In a preferred embodiment, the electrodes detect a cardiac signal from the surface of the heart and the signal is then recorded on a computer. The computer analyzes the signal data to determine a locus of atrial fibrillation i.e., where in the cardiac tissue the fibrillation is originating from. The computer then outputs a site in the cardiac tissue to be ablated based on the recorded signals from the electrodes.

### Common Mode Rejection

An embodiment of the invention provides a two-dimensional array of single electrodes arranged to facilitate measurement of electric potential in space over a tissue. Such an arrangement is referred to herein as a common mode rejection (CMR) electrode arrangement.

In a CMR electrode array, as opposed to being arranged in a stacked arrangement (i.e., pairs of electrodes), the electrodes are comprised of single electrodes, which may include microelectrodes, distributed across the array at known locations and each electrode is separated by a known distance. This simple embodiment allows for rapid computation of potentials across regions of a tissue. For example, electrodes in the CMR array may be useful in detecting signals of interest as bioelectrical signals propagate through tissue underneath the CMR array.

A "central" electrode in the array that detects the local activation signal works in conjunction with multiple "surrounding" contact electrodes that surround the central electrode on the array. As a local signal passes underneath the central electrode, a signal is concurrently recorded from both the central electrode and the surrounding electrodes. The signals can be entered into or directly recorded to a computer that averages the signals from the surrounding electrodes, and the resulting average is subtracted, by the computer, from the central electrode signal.

As a result, the computer is able to use signals detected from the CMR electrode array to accurately measure a given signal (e.g., an activation signal when used in cardiac tissue) by eliminating both far-field signal interference and near-field signal interreference from other electrodes on the array. As a result, a signal detected by the central electrode represents only the signal generated by the activation signal as it passes underneath the electrode. The CMR electrode array is thus able to leverage the benefits of unipolar electrodes and bipolar electrodes, while eliminating their drawbacks.

Accordingly, the use of simultaneously obtained electrode data according to the invention enables one to determine relative positions of measurements made by multiple electrodes in the construction of tissue mapping and the like (e.g., cardiac mapping).

For example, the CMR may be useful in cardiac applications wherein the CMR array may be positioned as a distribution of electrodes on the surface of the cardiac tissue so as to allow the computation of conduction velocity by a computer. The array can further be used to record signals in order for a computer to construct a map of cardiac rhythm and tissue properties, such as scarring. Based on the properties and signals detected, a computer can analyze the data and determine an optimal site for ablation to terminate or reduce the frequency of atrial fibrillation.

### Incorporation by Reference

References and citations to other documents, such as patents, patent applications, patent publications, journals, books, papers, web contents, have been made throughout this disclosure.

All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for detecting cardiac fibrillation, the method comprising: recording, via electrodes on a catheter comprising paddles with tunable conformance, signals from a cardiac tissue surface; entering the signals into a computer having resident therein software capable of determining a locus of atrial fibrillation in the cardiac tissue; and causing the computer to output a site for ablation.

2. The method of claim 1, wherein the catheter is tuned using wires.

3. The method of claim 2, wherein the wires control the ability of the catheter to conform.

4. The method of claim 3, wherein changing the durometer of the catheter causes the catheter to lie flat.

5. The method of claim 1, wherein electrode density on the catheter can be changed.

6. The method of claim 1, wherein the catheter can be tuned while in use.

7. A catheter able to be tuned to conform to the cardiac tissue surface.

8. The catheter of claim 7, wherein the catheter is tuned using wires.

9. The catheter of claim 8, wherein the wires control the ability of the catheter to conform.
